(19) United States
(12) Patent Application Publication
(10) Pub. No.: US 2026/0273010 A1
(43) Pub. Date: Sep. 17, 2026

(54) COMPOSITION FOR IMPROVING BLOOD CIRCULATION COMPRISING EXTRACT OF CHILI PEPPER BY-PRODUCT AS ACTIVE INGREDIENT

(71) Applicant: KOREA INSTITUTE OF ORIENTAL MEDICINE, Daejeon (KR)

(72) Inventors: KYUNGHO KIM, Daegu (KR); TAE IN KIM, Gyeongsangbuk-do (KR); YEON-JI KIM, Daegu (KR)

(21) Appl. No.: 19/471,858

(22) PCT Filed: Apr. 1, 2024
(86) PCT No.: PCT/KR2024/004157

§ 371 Date: Oct. 2, 2025

(30) Foreign Application Priority Data
Apr. 3, 2023 (KR) 10-2023-0043318

(51) Int. Cl. A61K 36/81; A61P 7/02

(52) U.S. Cl. CPC A61K 36/81; A61P 7/02; A61K 2236/331; A61K 2236/333

(57) ABSTRACT

A method for treating thrombosis includes administering a composition including an extract of chili pepper by-product as active ingredient to a subject in need thereof. Specifically, the extract of chili pepper by-product, for example, the extract of chili pepper leaf or chili pepper stem, exhibits excellent platelet aggregation inhibitory effects. In particular, the ethanol extract shows significantly stronger platelet aggregation inhibition compared to water extract. Therefore, the composition including an extract of chili pepper by-product as active ingredient can be advantageously used as a therapeutic agent for thrombosis, an anti-thrombosis functional health food, or as a material for anti-thrombosis oriental herbal medicine composition.

### BACKGROUND

### 1. Technical Field

The present invention relates to a composition for improving blood circulation including an extract of chili pepper by-product as active ingredient.

This study was carried out with the support of the National Research Foundation of Korea under the Science and Engineering Academic Research Infrastructure Construction Project (Project No.: 2021R1I1A2040301 (NSN2114180)).

### 2. Background Art

Blood circulation is primarily regulated by the plasma and blood cells (i.e., red blood cells and platelets) that make up the blood. These components maintain the homeostasis of blood flow and support normal hemostasis and protective functions at sites of vascular injury or inflammation, thereby sustaining normal physiological functions of the human body. However, excessive activation of coagulation factors in plasma, increased platelet aggregation, or abnormal deformability of red blood cells can disrupt blood flow homeostasis, leading to circulatory disorders and cardiovascular diseases such as arteriosclerosis and brain stroke.

In normal blood vessels, the activation and inhibition of hemostatic mechanisms are balanced, maintaining homeostasis. Excessive hemostatic activity and thrombus formation, however, impede blood flow, causing circulatory abnormalities and leading to lesions such as thrombus.

Thrombus-related diseases, which account for approximately 39% of global deaths, are increasingly prevalent in South Korea due to Westernized diets, excessive stress, and an aging population. In particular, the rising incidence, prevalence, and number of diagnoses in developing countries such as China and India are expected to contribute to the growth.

Currently used thrombolytic agents include streptokinase, urokinase, and tissue plasminogen activator (t-PA), which primarily dissolve thrombus through an indirect mechanism by activating plasminogen in vivo to plasmin.

Streptokinase and urokinase are exogenous enzymatic agents that convert plasminogen to plasmin, but they have side effects such as fever, allergic reactions, local hemorrhage, and hypotension. In addition, they are classified as very expensive therapeutic drugs and are limited to treatment purposes, being unsuitable for the prevention of thrombus formation in the human body.

Consequently, research has been actively conducted on natural plant-derived substances that are safe and exhibit excellent blood circulation-enhancing effects. Compared to synthetic substances that may cause side effects, developing the formulations based on traditionally validated natural substances to improve blood circulation is considered more desirable.

Meanwhile, prior art related to the blood circulation-enhancing effects of natural products includes Korean Patent Registration No. 2183915, which discloses a composition for improving blood circulation including a combined extract of Angelica root, Cynanchum wilfordii, and Ginkgo biloba leaves as an active ingredient, and Korean Patent Registration No. 1352591, which discloses a composition for improving blood circulation including an extract of thistle. However, to date, no disclosure has been made regarding the composition for improving blood circulation including an extract of chili pepper by-product, specifically an extract of chili pepper leaf or chili pepper stem, as active ingredient that is described in the present invention.

### SUMMARY

The present invention is devised in response to the above-described needs, and provides a composition for improving blood circulation including an extract of chili pepper by-product, specifically an extract of chili pepper leaf or chili pepper stem, as active ingredient. The present invention is completed by finding the platelet aggregation-inhibitory effect of the extract of chili pepper leaf or chili pepper stem.

To solve the problems that are described above, the present invention provides a pharmaceutical composition for preventing or treating thrombosis including an extract of chili pepper by-product as active ingredient.

The present invention further provides a functional health food composition for anti-thrombosis including an extract of chili pepper by-product as active ingredient.

The present invention further provides a feed additive for anti-thrombosis including an extract of chili pepper by-product as active ingredient.

The present invention further provides a veterinary composition for preventing or treating thrombosis including an extract of chili pepper by-product as active ingredient.

The present invention further provides an oriental herbal medicine composition for anti-thrombosis including an extract of chili pepper by-product as active ingredient.

The present invention still further provides a platelet aggregation inhibitor including an extract of chili pepper by-product as active ingredient.

The present invention relates to a composition for improving blood circulation including an extract of chili pepper by-product as active ingredient. Specifically, the extract of chili pepper by-product, namely the extract of chili pepper leaf or chili pepper stem, exhibits excellent platelet aggregation inhibitory effects. In particular, the ethanol extract shows significantly stronger platelet aggregation inhibition compared to water extract. Therefore, the composition of the present invention including an extract of chili pepper by-product as active ingredient can be advantageously used as a therapeutic agent for thrombosis, an anti-thrombosis functional health food, or as a material for anti-thrombosis oriental herbal medicine composition.

### BRIEF DESCRIPTION OF THE DRAWINGS

FIG. 1 shows the results of determining the degree of platelet aggregation following treatment with the chili pepper by-product extract according to the present invention. Platelet aggregation was induced by treatment with collagen. Vehicle represents the control group not treated with any test sample (i.e., extract). ** and *** indicate that the platelet aggregation in the test sample-treated group was statistically significantly reduced compared to the control (Vehicle), with ** representing p<0.01 and *** representing p<0.001. # and ## indicate that platelet aggregation in the chili pepper leaf ethanol extract-treated group was statistically significantly lower than that in the chili pepper stem ethanol extract-treated group, with # representing p<0.05 and ## representing p<0.01.

FIGS. 2A to 2D show the results of determining the inhibitory effect of the chili pepper leaf extract of the present invention on platelet aggregation induced by different platelet aggregation inducing factors: thrombin (FIG. 2A), collagen (FIG. 2B), CRP (collagen-related peptide) (FIG. 2C), and U46619 (Thromboxane A2 agonist) (FIG. 2D). Vehicle represents the control group not treated with any test sample. *, **, and *** indicate statistically significant reductions in platelet aggregation in the test sample-treated group compared to the control (Vehicle), with * representing p<0.05, ** representing p<0.01, and *** representing p<0.001.

FIGS. 3A and 3B show the results of determining the degree of platelet activation following treatment with the chili pepper leaf extract of the present invention, assessed by P-selectin (FIG. 3A) and integrin αIIbβ3 (FIG. 3B). Platelet activation was induced by treatment with CRP (collagen-related peptide). “−” represents the negative control with no treatment, and Vehicle represents the control treated with CRP only. *, **, and *** indicate that P-selectin or integrin αIIbβ3 activation in the test sample-treated group was statistically significantly reduced compared to the control (Vehicle), with * representing p<0.05, ** representing p<0.01, and *** representing p<0.001.

FIGS. 4A to 4D show the results of determining the inhibitory effect of the chili pepper leaf extract on ATP secretion induced by platelet aggregation inducing factors: thrombin (FIG. 4A), collagen (FIG. 4B), CRP (collagen-related peptide) (FIG. 4C), and U46619 (Thromboxane A2 agonist) (FIG. 4D). Vehicle represents the control treated only with the platelet aggregation inducing factors. *** indicates that ATP secretion in the test sample-treated group was statistically significantly reduced compared to the control (Vehicle), with p<0.001.

FIG. 5 shows the results of determining the changes in TXB₂ concentration following treatment with the chili pepper leaf extract of the present invention. Platelet activation was induced by treatment with CRP (collagen-related peptide). Vehicle represents the control treated with CRP only. ** and *** indicate that TXB₂ concentration in the test sample-treated group was statistically significantly reduced compared to the control (Vehicle), with ** representing p<0.01 and *** representing p<0.001.

FIGS. 6A and 6B show the results of determining the effects of the chili pepper leaf extract on platelet Ca²⁺ release (FIG. 6A) and intracellular Ca²⁺ influx (FIG. 6B). CRP (collagen-related peptide) was used to increase Ca²⁺ release and influx. Vehicle represents the control treated with CRP only. *, **, and *** indicate that Ca²⁺ release or influx in the test sample-treated group was statistically significantly reduced compared to the control (Vehicle), with * representing p<0.05, ** representing p<0.01, and *** representing p<0.001.

### DETAILED DESCRIPTION

To achieve the object of the present invention, the present invention provides a pharmaceutical composition for preventing or treating thrombosis including an extract of chili pepper by-product as active ingredient.

The chili pepper by-product is preferably a chili pepper leaf or a chili pepper stem, more preferably a chili pepper leaf, but it is not limited thereto.

Extraction solvent for the extract may be water, C1 to C4 lower alcohol, or a mixture thereof, preferably water or ethanol, and more preferably ethanol, but it is not limited thereto.

In one embodiment of the present invention, the composition is characterized in that it inhibits platelet aggregation induced by one or more factors selected from thrombin, collagen, CRP (collagen-related peptide), and TXA₂ agonists (thromboxane A2 agonist). Thus, the extract of chili pepper by-product can inhibit excessive platelet aggregation and thereby improve blood circulation.

The composition of the present invention is preferably formulated in one of the dosage forms selected from capsule, powder, granule, tablet, suspension, emulsion, syrup, and aerosol, but it is not limited thereto.

In addition to the active ingredient, the composition of the present invention may further include pharmaceutically acceptable carriers, excipients, or diluents, and can be formulated in various oral or parenteral forms. When formulating, commonly used excipients or carriers such as fillers, bulking agents, binders, humectants, disintegrants, or surfactants may be employed. For oral solid preparations, suitable forms include capsules, powders, granules, tablets, and pills, which can be prepared by mixing one or more compounds with at least one excipient, such as starch, calcium carbonate, sucrose, lactose, or gelatin. In addition to simple excipients, lubricants such as magnesium stearate or talc may be included. For oral liquid preparations, suitable forms include suspensions, emulsions, syrups, and aerosols, which may contain conventional diluents such as water or liquid paraffin, as well as additional excipients, e.g., humectants, sweeteners, flavoring agents, or preservatives. For parenteral administration, suitable formulations include sterile aqueous solutions, non-aqueous solvents, suspensions, emulsions, lyophilized formulations, and suppositories. Non-aqueous solvents and suspension vehicles may include propylene glycol, polyethylene glycol, vegetable oils such as olive oil, and injectable esters such as ethyl oleate. Suppository bases may include Witepsol, macrogol, Tween 61, cocoa butter, laurin fat, or glycerol gelatin. For parenteral administration, routes such as topical application, intraperitoneal, rectal, intravenous, intramuscular, subcutaneous, intrauterine epidural, or intracerebrovascular injection are preferably selected

The pharmaceutical composition according to the present invention is administered in a pharmaceutically effective amount. In the present invention, a “pharmaceutically effective amount” refers to an amount sufficient to treat a disease with a reasonable benefit/risk ratio applicable to medical treatment. The effective amount can be determined based on factors that are well known in the medical field, including the type and severity of the patient's disease, drug activity, sensitivity to the drug, administration time, route of administration and excretion rate, treatment period, concomitant medications, and other relevant factors. The composition of the present invention can be administered as a single therapeutic agent or in combination with other therapeutic agents, either sequentially or simultaneously, and may be given as a single or multiple dose. Considering all the aforementioned factors, it is important to administer an amount that achieves maximum effect with minimum side effects, which can be readily determined by those skilled in the art.

The dosage of the composition of the present invention may vary depending on the patient's body weight, age, sex, health condition, diet, time and method of administration, excretion rate, and disease severity. The composition of the present invention can be used either alone or in combination with surgery, radiation therapy, hormone therapy, chemotherapy, or methods using biological response modifiers.

The present invention further provides a functional health food composition for anti-thrombosis including an extract of chili pepper by-product as active ingredient.

The composition is characterized by its ability to inhibit excessive platelet aggregation, thereby improving blood circulation.

The composition can be preferably formulated in any one of the forms selected from powder, granule, pills, tablet, capsule, candy, syrup, and beverage, but it is not limited thereto.

When the functional health food composition of the present invention is used as a food additive, the active ingredient may be added directly or used together with other foods or food components, and can be used appropriately according to conventional methods. The mixing amount of the active ingredient can be suitably determined according to its intended purpose (prevention, health maintenance, or therapeutic treatment). Generally, when producing food or beverages, the composition of the present invention is added in an amount of 15 parts by weight or less, preferably 10 parts by weight or less, relative to the raw materials. However, for long-term intake for health or hygiene purposes, the amount may be below this range, and since there are no safety issues, the active ingredient can also be used in amounts exceeding the above range. The type of food is not particularly limited. Examples of foods to which the extract or its fractions can be added include meat, sausages, bread, chocolate, candies, snacks, confectioneries, pizza, ramen, other noodles, gums, dairy products including ice cream, various soups, beverages, teas, drink preparations, alcoholic beverages, and vitamin complexes, encompassing all those within conventional meanings of functional health foods.

When the composition of the present invention is used as a health beverage, it may contain various flavoring agents or natural carbohydrates as additional ingredients, similar to conventional beverages. The aforementioned natural carbohydrates include monosaccharides such as glucose and fructose, disaccharides such as maltose and sucrose, polysaccharides such as dextrin and cyclodextrin, and sugar alcohols such as xylitol, sorbitol, and erythritol. Sweeteners may include natural sweeteners such as thaumatin and stevia extract, or synthetic sweeteners such as saccharin and aspartame. The proportion of the natural carbohydrates is generally about 0.01 g to 0.04 g, preferably about 0.02 g to 0.03 g per 100 g of the composition of the present invention. The composition may further contain various nutrients, vitamins, electrolytes, flavoring agents, colorants, pectic acid and its salts, alginic acid and its salts, organic acids, protective colloid agents, pH adjusters, stabilizers, preservatives, glycerin, alcohol, and carbonating agents used in carbonated beverages. In addition, the composition of the present invention may contain fruit pulp for the production of natural fruit juice, fruit juice beverages, and vegetable beverages. These components can be used individually or in combination. The proportion of these additives is generally not critical, but typically ranges from 0.01 to 0.1 parts by weight per 100 parts by weight of the composition of the present invention.

The present invention further provides a feed additive for anti-thrombosis including an extract of chili pepper by-product as active ingredient.

The feed additive of the present invention corresponds to a supplementary feed under the Feed Control Act. In the present invention, the term “feed” may refer to any natural or artificial formulated feed, single-meal feed, or a component of such a meal that is suitable for animals to eat, ingest, and digest. The type of feed is not particularly limited, and any feed commonly used in the relevant field can be employed. Non-limiting examples of the feed include plant-based feeds such as grains, root and tuber crops, by-products of food processing, algae, fibers, pharmaceutical by-products, oils, starches, oil cakes, or grain by-products; and animal-based feeds such as proteins, minerals, fats, single-cell proteins, animal plankton, or food scraps. These feeds can be used either alone or in combination with two or more types thereof.

The present invention further provides a veterinary composition for preventing or treating thrombosis including an extract of chili pepper by-product as active ingredient.

The veterinary composition of the present invention may further include suitable excipients and diluents according to conventional methods. Excipients and diluents that can be included in the veterinary composition of the present invention include lactose, dextrose, sucrose, sorbitol, mannitol, xylitol, erythritol, maltitol, starch, acacia gum, alginate, gelatin, calcium phosphate, calcium silicate, cellulose, methyl cellulose, amorphous cellulose, polyvinylpyrrolidone, water, methyl hydroxybenzoate, propyl hydroxy benzoate, talc, magnesium stearate, cetanol, stearyl alcohol, liquid paraffin, sorbitan monostearate, polysorbate 60, methylparaben, propylparaben, and mineral oil.

The veterinary composition according to the present invention may additionally include fillers, anti-caking agents, lubricants, humectants, flavoring agents, emulsifiers, preservatives, and the like. The veterinary composition according to the present invention can be formulated using methods that are well known in the art to provide rapid, sustained, or delayed release of the active ingredient after administration to an animal. The dosage form may include powders, granules, tablets, capsules, suspensions, emulsions, solutions, syrups, aerosols, soft or hard gelatin capsules, suppositories, sterile injectables, or sterile topical formulation

The effective amount of the veterinary composition according to the present invention can be appropriately selected depending on the individual animal. It may be determined based on factors well known in the physiological and veterinary fields, including the severity of the disease or condition, the age, body weight, health status, or sex of the animal, the sensitivity of the animal to the active ingredient of the present invention, the route and duration of administration, and other factors such as the combination or concurrent use of other compositions.

The present invention further provides an oriental herbal medicine composition for anti-thrombosis including an extract of chili pepper by-product as active ingredient.

The oriental herbal medicine composition of the present invention refers to a preparation made according to traditional Korean medicine prescriptions, but it is not limited thereto.

An oriental herbal medicine formulation containing the oriental herbal medicine composition according to one embodiment of the present invention may also be provided. The oriental herbal medicine formulation may simply include the oriental herbal medicine composition and is not limited to any specific form. For example, the oriental herbal medicine formulation may be in the form of a liquid preparation, pill, tablet, granule, capsule, health food, or health drink.

The present invention still further provides a platelet aggregation inhibitor including an extract of chili pepper by-product as active ingredient.

In one embodiment of the present invention, the active ingredient has the effect of inhibiting platelet aggregation induced by one or more factors selected from thrombin, collagen, CRP (collagen-related peptide), and TXA₂ agonists (thromboxane A2 agonist).

Hereinafter, the present invention will be described in greater detail with reference to the following preparation examples and experimental examples. These preparation and experimental examples are provided solely to illustrate more specifically the present invention, and it will be apparent to those skilled in the art that the scope of the present invention is not limited thereby.

### EXAMPLES

### Preparation Example 1. Preparation of Extract of Chili Pepper By-Product

1) Preparation of 30% (v/v) Ethanol Extract of Chili Pepper Leaves

The raw chili pepper leaves were weighed, and 30% (v/v) ethanol, 10 times the weight of the raw chili pepper, was added and allowed to soak for 1 hour. After soaking, the mixture was extracted twice using a reflux cooling extraction method at 90° C. to 100° C. The resulting extract was filtered using filter paper, and the filtrate was concentrated under reduced pressure and then freeze-dried to give the 30% ethanol extract of chili pepper leaves in powder form. The extract powder was dissolved in DMSO for use in experiments.

### 2) Preparation of Water Extract of Chili Pepper Leaves

The raw chili pepper leaves were weighed, and distilled water, 10 times the weight of the raw chili pepper, was added and allowed to soak for 1 hour. The mixture was then extracted twice at 100° C. for 2 hours and 30 minutes using an oriental herbal medicine extractor. After extraction, the solution was filtered through a mesh, and the filtrate was freeze-dried to give the hot water extract of chili pepper leaves in powder form. The extract powder was dissolved in DMSO for use in experiments.

3) Preparation of 30% (v/v) Ethanol Extract of Chili Pepper Stems

The raw chili pepper stems were weighed, and 30% (v/v) ethanol, 10 times the weight of the raw chili pepper, was added and allowed to soak for 1 hour. After soaking, the mixture was extracted twice using a reflux cooling extraction method at 90° C. to 100° C. The resulting extract was filtered using filter paper, and the filtrate was concentrated under reduced pressure and then freeze-dried to give the 30% ethanol extract of chili pepper stems in powder form. The extract powder was dissolved in DMSO for use in experiments.

### 4) Preparation of Water Extract of Chili Pepper Stems

The raw chili pepper stems were weighed, and distilled water, 10 times the weight of the raw chili pepper, was added and allowed to soak for 1 hour. The mixture was then extracted twice at 100° C. for 2 hours and 30 minutes using an oriental herbal medicine extractor. After extraction, the solution was filtered through a mesh, and the filtrate was freeze-dried to give the hot water extract of chili pepper stems in powder form. The extract powder was dissolved in DMSO for use in experiments.

### Example 1. Determination of Platelet Aggregation Inhibitory Effect

### 1) Platelet Aggregation Inhibitory Effect of Chili Pepper By-Product Extract

Mouse platelets were treated with the chili pepper by-product extract at various concentrations for 15 minutes at 37° C. Platelet aggregation was then induced by treating the platelets with a platelet aggregation factor, collagen (1 μg/mL), to activate them. Thereafter, the degree of platelet aggregation was measured and analyzed using a 4-channel platelet lumi-aggregometer (Chronolog Corp, Havertown, PA).

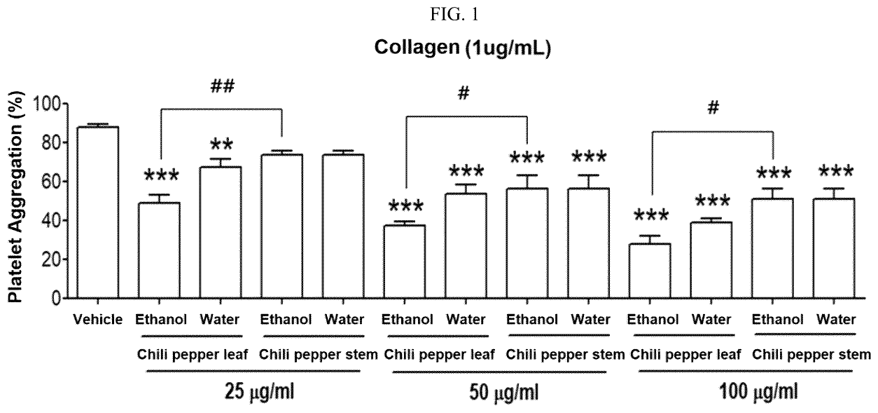

As shown in FIG. 1, the extract of chili pepper by-product of the present invention, specifically the extract of chili pepper leaf or chili pepper stem, exhibited a concentration-dependent inhibitory effect on collagen-induced platelet aggregation.

The ethanol extract showed a significantly stronger inhibitory effect compared to the water extract, and in particular, the ethanol extract of chili pepper leaf demonstrated superior platelet aggregation inhibition compared to the ethanol extract of chili pepper stem. Therefore, subsequent experiments were conducted using the ethanol extract of chili pepper leaf as the test sample.

2) Platelet Aggregation Inhibitory Effect of Chili Pepper Leaf Extract with Various Platelet Aggregation Inducing Factors

To evaluate the effect of the chili pepper leaf extract on platelet aggregation induced by various platelet aggregation inducing factors, mouse plasma was treated with thrombin, collagen, CRP (collagen-related peptide), or U46619 (Thromboxane A2 agonist) to activate platelet aggregation. After the pre-treatment with different concentrations of chili pepper leaf ethanol extract, the degree of platelet aggregation was measured and analyzed using a 4-channel platelet lumi-aggregometer (Chronolog Corp., Havertown, PA).

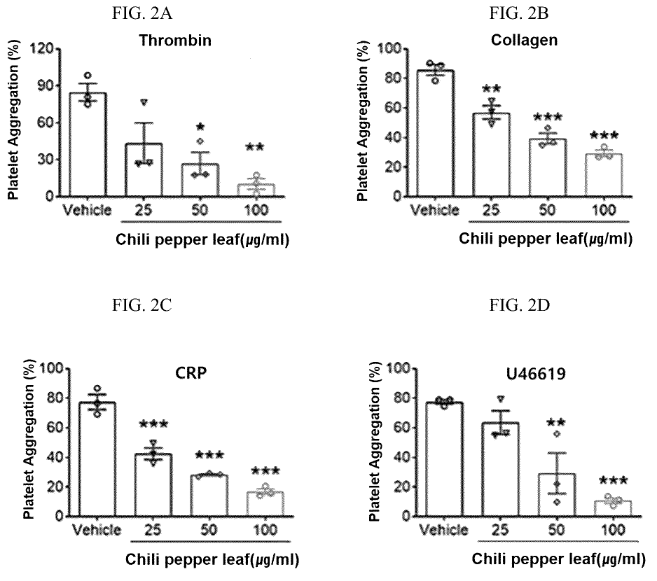

As shown in FIGS. 2A to 2D, the chili pepper leaf extract-treated group exhibited a concentration-dependent and significant inhibition of platelet aggregation compared to the control group (vehicle).

### Example 2. Evaluation of Platelet Activation Inhibitory Effect of Chili Pepper Leaf Extract

After a treatment with the chili pepper leaf extract, the activation level of platelet α-granules, key biomarkers involved in the positive feedback cycle of platelet activation—specifically P-selectin and integrin αIIBβ3—was examined using flow cytometry analysis.

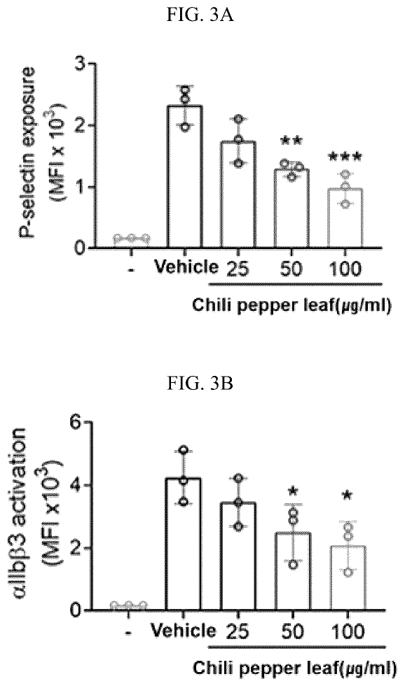

As shown in FIGS. 3A and 3B, a treatment with the chili pepper leaf extract inhibited the activation of platelet α-granule P-selectin and αIIBβ3 integrin in mouse platelets in a concentration-dependent manner compared to the control group (vehicle).

### Example 3. Evaluation of Platelet Aggregation Inhibiting Activity of Chili Pepper Leaf Extract Via ATP Secretion Measurement

During platelet aggregation, ATP is secreted from platelets and activates intrinsic receptors on the platelet outer membrane (P2Y1, P2Y12) through autocrine and paracrine signaling, thereby promoting platelet aggregation. ATP secretion and activation are therefore recognized as key mechanisms in thrombus formation. Thus, in order to assess the effect of chili pepper leaf extract on ATP secretion, platelet aggregation was first induced using thrombin, CRP, collagen, or U46619, and then the chili pepper leaf extract was applied at various concentrations. Thereafter, ATP secretion amount was quantified using a luciferin/luciferase reagent (Chronolog Corp.).

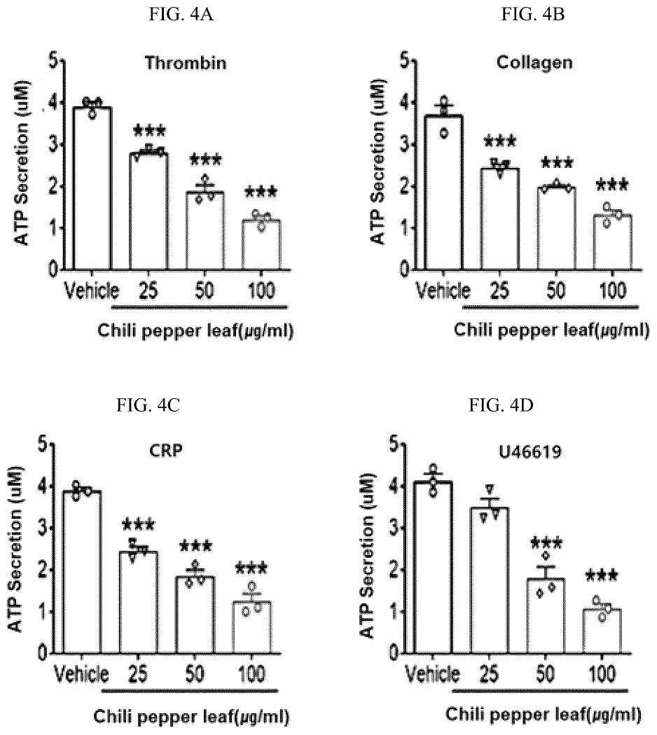

As shown in FIGS. 4A to 4D, a treatment with the chili pepper leaf extract inhibited platelet ATP secretion in a concentration-dependent manner compared to the control group (vehicle).

### Example 4. Evaluation of Platelet Aggregation Inhibiting Activity pf Chili Pepper Leaf Extract Via TXB₂ Generation Measurement

During platelet aggregation, TXB₂ is produced and released, activating intrinsic TP receptors on the platelet outer membrane through autocrine and paracrine signaling, thereby promoting platelet aggregation. TXB₂ generation and activation are thus recognized as key mechanisms in thrombus formation. In order to examine the effect of chili pepper leaf extract on TXB₂ generation, platelet aggregation activity was first enhanced using CRP. Chili pepper leaf extract was then applied at various concentrations, and the level of TXB₂ was measured using a TXB₂ generation assay kit.

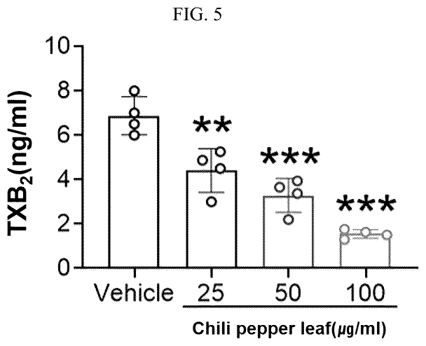

As shown in FIG. 5, a treatment with the chili pepper leaf extract inhibited TXB₂ generation in a concentration-dependent manner compared to the control group (vehicle).

### Example 5. Evaluation of Effect of Chili Pepper Leaf Extract on Platelet Ca²⁺ Mobilization

To investigate the mechanism by which the chili pepper leaf extract inhibits platelet activation, intracellular Ca²⁺ mobilization—a key step in platelet activation—was examined. Mouse platelets (1×10⁸ cells/mL) were treated with vehicle (control) or various concentrations of the chili pepper leaf extract at 37° C. for 10 minutes. The pre-treated platelets were then stained with a Ca²⁺ staining agent (FLIPR Calcium 5 Assay kit) in the dark at 37° C. for 30 minutes, and then platelet activation was induced by using 0.5 μg/mL CRP. Ca²⁺ release and influx concentrations inside the platelets were measured using a fluorescence plate reader (Spectramax i3, Molecular Devices) at an excitation wavelength of 485 nm and emission wavelength of 525 nm.

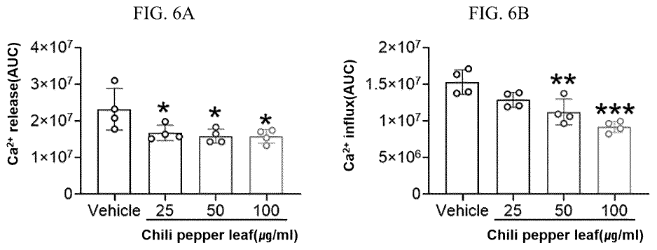

As shown in FIGS. 6A and 6B, upon the platelet cell activation caused by CRP treatment, the intracellular Ca²⁺ influx and release were markedly increased, whereas the pre-treatment with the chili pepper leaf extract of the present invention reduced both Ca²⁺ influx and release. Based on these results, it was found that the chili pepper leaf extract can modulate CRP-induced Ca²⁺ mobilization in platelets.

1-13. (canceled)

14. A method for treating thrombosis, the method comprising:
administering a composition comprising an extract of chili pepper by-product as active ingredient to a subject in need thereof.

15. The method of claim 14, wherein the chili pepper by-product is a chili pepper leaf and/or a chili pepper stem.

16. The method of claim 14, wherein an extraction solvent for the extract is water, C1 to C4 lower alcohol, or a mixture thereof.

17. The method of claim 14, wherein the composition is a pharmaceutical composition further comprising a pharmaceutically acceptable carrier, an excipient, and/or a diluent.

18. The method of claim 14, wherein the composition is formulated in any one form selected from capsule, powder, granule, tablet, suspension, emulsion, syrup, and aerosol.

19. The method of claim 14, wherein the composition is a functional health food composition formulated in powder, granule, pill, tablet, capsule, candy, syrup, and/or beverage.

20. The method of claim 14, wherein the composition is administered as a feed additive.
